(19) United States
(12) Patent Application Publication
(10) Pub. No.: US 2004/0068185 A1
(43) Pub. Date: Apr. 8, 2004

(54) Ultrasonic diagnostic imaging system with articulating display handle

(76) Inventors: Elizabeth A. Marshall, Bothell, WA (US); Daryl E. Schilke, Seattle, WA (US); Ulrich Rankers, Livermore, CA (US)

Correspondence Address: ATL ULTRASOUND, P.O. BOX 3003, 22100 BOTHELL EVERETT HIGHWAY, BOTHELL, WA, 98041-3003, US

(21) Appl. No.: 10/267,152

(22) Filed: Oct. 8, 2002

(51) Int. Cl. A61B 8/00

(52) U.S. Cl. 600/437

(57) ABSTRACT

A cart-borne ultrasonic diagnostic imaging system includes an image display which is mounted on an articulating mechanism on the cart. The articulating mechanism enables the image display to be moved relative to the cart from a nominal centered and forward-facing position to the side of the cart and to be rotated toward the side of the cart. The image display can also be articulated to face upward or downward from its nominal position. The image display includes a handle mounted on the front of the display which can be gripped by the user to move the display rotationally, forward and back, left and right, and to face upward or downward. Either a CRT display or a flat panel display can be articulated in this manner.

This invention relates to ultrasonic diagnostic imaging systems and, in particular, to ultrasonic diagnostic imaging systems with displays that can be articulated for ease and comfort of viewing.

Designs of ultrasound systems are increasingly taking the comfort and convenience of the user and patient into consideration. These efforts have been stimulated by reports of repetitive stress injuries and by the desire to provide additional comfort and convenience for those using the ultrasound system, including both the operator and the patient. One component of the ultrasound system which is amenable to such designs is the display device on which the diagnostic image is displayed. As the operator is guiding the ultrasound probe over the body of the patient to acquire the anatomy of interest in the field of view of the probe, the operator is constantly watching the image produced by the probe on the system display. To do this comfortably and effectively, the operator needs to position the patient, the operator, and the display in related positions that enable the anatomy of interest to be effectively scanned while the operator watches the ultrasound image on the display. This procedure is aided when the display device, which may be a CRT monitor or a flat-panel display, can be easily moved to the desired viewing position.

To enable the user to adjust the monitor position, some ultrasound systems mount the monitor on the articulation mechanism conventionally found on many computer monitors. These mechanisms include a base mount on which the monitor can swivel about a vertical pivot axis, and which permits the monitor to be rocked about a horizontal axis so as to face more upward or downward toward the operator. However, the patient may want to see the image on the display from time to time, or the ultrasonographer may want to show the image to the patient. To show the patient the live image, the ultrasonographer will have to position the monitor toward the patient with one hand, while continuing to scan with the probe with the other hand. Accordingly, the display should be designed to enable the display device, either a CRT monitor or flat panel display, to be easily moved to a new viewing position with one hand while scanning a patient.

In accordance with the principles of the present invention, a cart-borne ultrasound system includes an articulating mounting device for the image display. The display device includes a handle or other control surface which can be gripped by the ultrasonographer with one hand. The articulating mounting device allows the display device to be repositioned by the ultrasonographer with one hand while the ultrasonographer continues to scan the patient.

In the drawings:

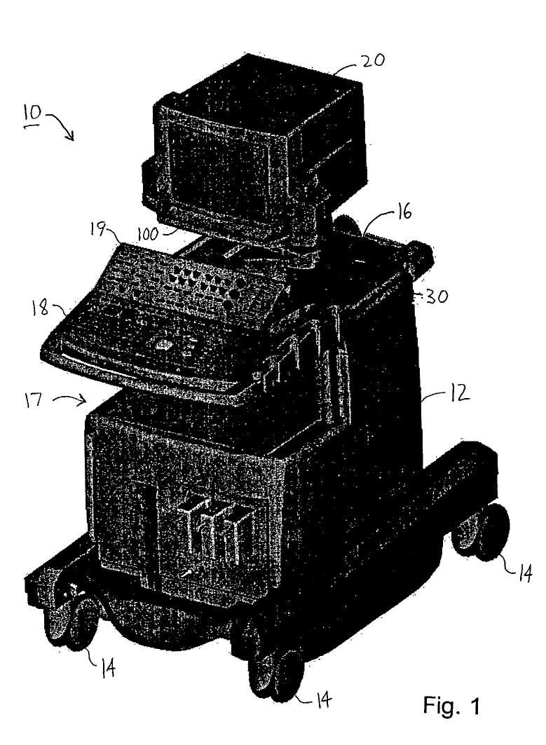

FIG. 1 illustrates a cart-borne ultrasound system of the present invention in a forward perspective view;

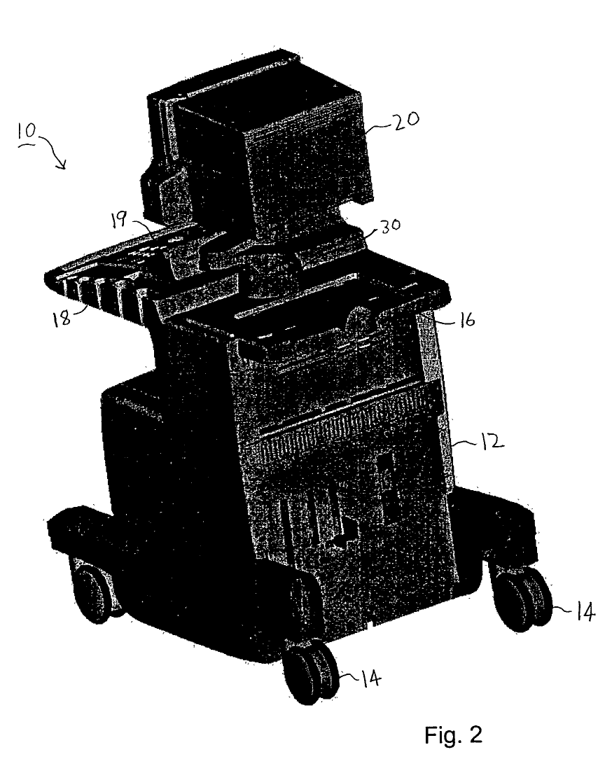

FIG. 2 illustrates a cart-borne ultrasound system of the present invention in a rearward perspective view;

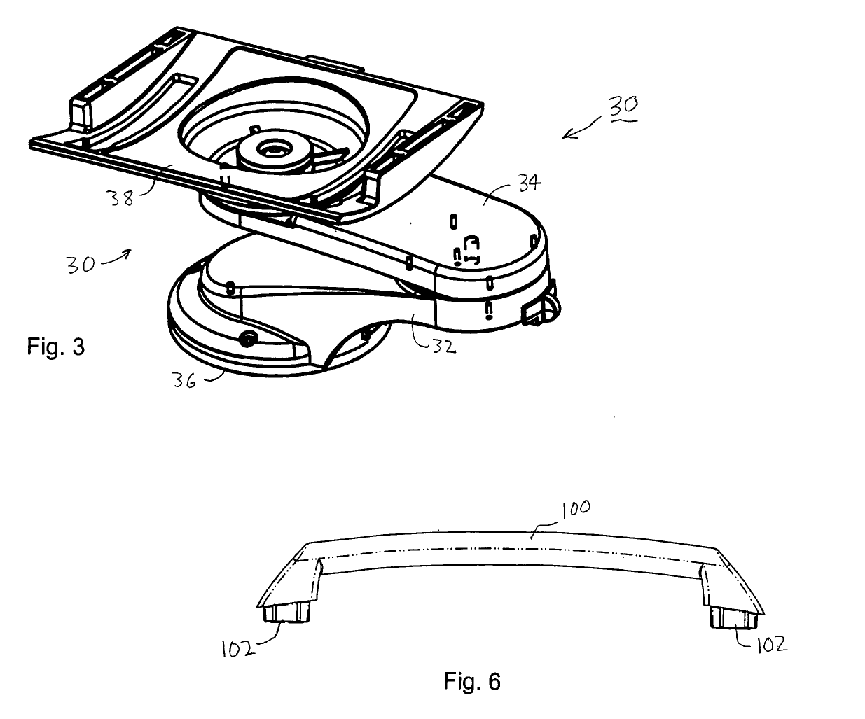

FIG. 3 is a perspective view of an articulating display mount constructed in accordance with the principles of the present invention;

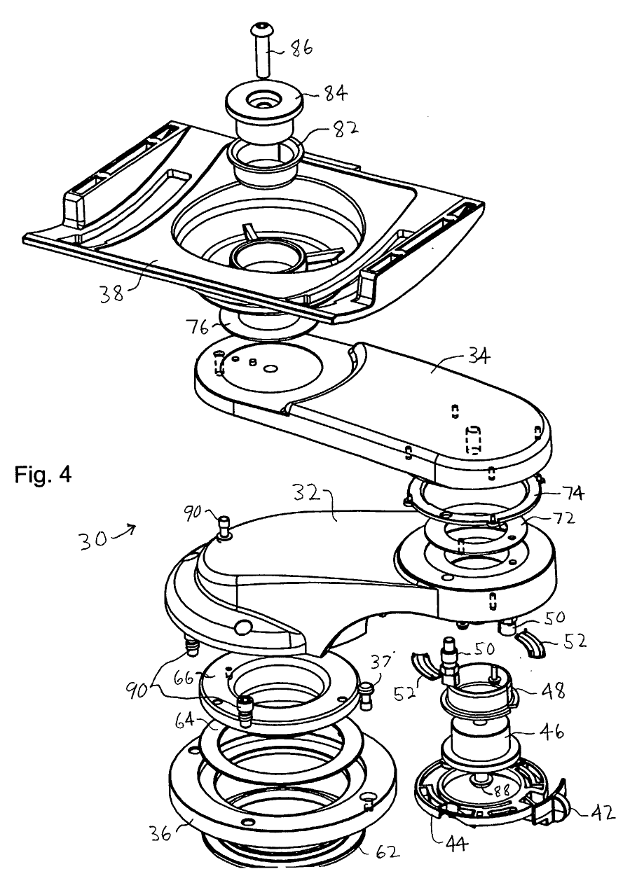

FIG. 4 is an exploded perspective view of the articulating display mount of FIG. 3;

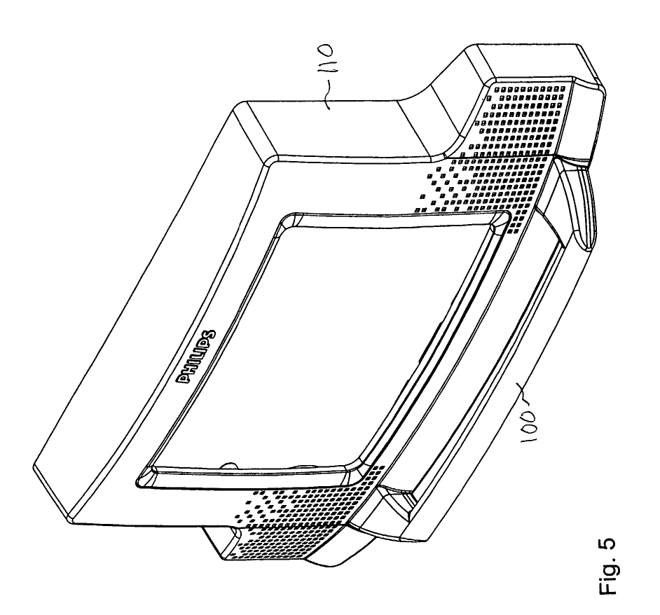

FIG. 5 is a perspective view of a display bezel for a preferred embodiment of the present invention;

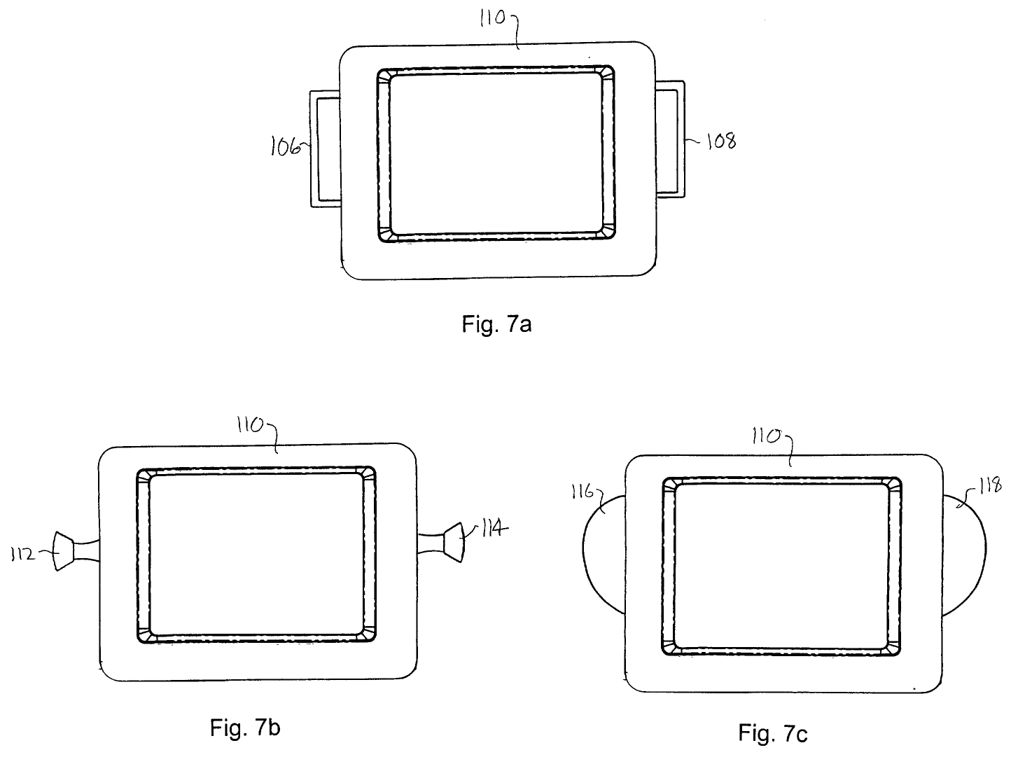

FIG. 6 is a plan view of the display handle used on the display bezel of FIG. 5; and

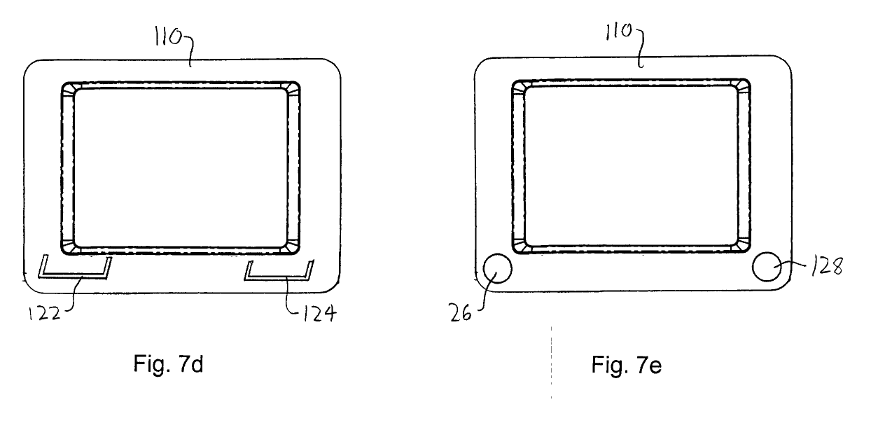

FIGS. 7a-7e illustrate embodiments of the present invention with different display handles and handle positions.

Referring first to FIGS. 1 and 2, a cart-borne ultrasound system 10 is shown in forward and rearward perspective views. The ultrasound system cart includes a main body 12 which contains the electronics of the system, including a card cage with specially designed ultrasound circuitry such as beamformers and signal and image processors and associated power supplies. The cart is mounted on wheels or casters 14. The cart has a top surface in the front which forms an accessory bay 17 in which accessory devices such as a printer can be installed. Above the accessory bay 17 is a control panel 18 which the operator uses to set up and control an ultrasound examination. The illustrated control panel has a rear portion 19 which is inclined upward so as to put the buttons and switches on that portion of the control panel within easy reach of the operator. In a preferred embodiment the control panel is movable so that it can be raised and lowered and be more comfortable for both sitting and standing operators.

The cart also has a rear top surface 16 which accommodates other accessory devices such as a video recorder.

In accordance with the principles of the present invention the ultrasound system 10 has a monitor 20 located above the control panel. The monitor 20 is mounted on an articulating mechanism 30 which allows the monitor to be rotated and moved to a comfortable viewing position for the operator and/or the patient. The ultrasound system display may alternatively comprise a flat panel LCD or plasma display instead of the CRT monitor shown in FIGS. 1 and 2. The articulating mechanism 30 allows the monitor to be swiveled and repositioned without striking the raised portion 19 of the control panel or any accessory device located on the rear surface 16 of the cart.

The display articulating mechanism 30 is shown in an enlarged perspective view in FIG. 3. The embodiment there shown has a mount plate 36 at the bottom which can be secured to the top of the ultrasound system cart as described below. The mechanism 30 has a lower articulation arm 32 which is pivotally mounted on the mount plate 36. The lower articulation arm is a rigid member which is inclined upward at approximately a 16° angle which enables the articulating mechanism and display device to clear the raised portion 19 of the control panel 19 when the control panel is fully elevated and the monitor 20 is rotated forward over the control panel. In a constructed embodiment the lower arm provides an elevation of approximately three inches above the top surface of the cart. At its upper end the lower articulation arm 32 is pivotally connected to an upper articulation arm 34. At its other end the upper articulation arm 34 is pivotally connected to a monitor tilt and swivel base 38. The inclined angle of the lower articulation arm also prevents development of a pinch point between the lower arm and the top surface of the cart and between the lower and upper arms when the two arms overlap, which would not be the case if the arms were flush with each other or the lower arm were flush with the surface of the cart. The mount plate, lower articulation arm, and upper articulation arm can be hollow members which enable the passage of a power cord and cable to pass from the main body of the ultrasound system cart, through the pivot joints and arms, and to a monitor or flat panel display located on the tilt and swivel base. Alternatively the cord and cable can be loosely located about the articulating mechanism to enable the display to articulate freely without binding or tangling the cord and cable.

FIG. 4 is an exploded view of the articulating mechanism 30. The mount plate 36 is sandwiched by two bearing washers 62 and 64 which are conventional bearing washers with smooth Teflon®-like surfaces. Compression of the mount plate and bearing washers is maintained by a retainer 66, which is mounted to the top of the ultrasound system cart. The lower articulation arm 32 is mounted by bolts 90 to the mount plate 36. When the lower articulation arm is pivoted relative to the cart, the mount plate rotates between the bearing washers 62 and 64, with the retainer 66 preloading the clamping force (rotational friction) of this lower joint of the articulating mechanism 30.

The mount plate 36 has a screw 37 extending downward from the mount plate which travels in a circular trough formed in the top surface of the cart. The length of this trough defines a range of movement and end stops which restrict the travel of the screw 37 and thereby the angle of rotation of the lower joint to a predetermined angle. In a constructed embodiment the range of rotation used is 210°, which does not allow the lower articulation arm 32 to swing over the front-most 150° of a full circular range of rotation. This choice of pivot range prevents the lower articulation arm to swing fully to the front of the system where it might otherwise impact the upper portion 19 of the control panel. The lower articulation arm 32 is allowed to swing completely over the rear surface 16 of the cart, where the upward inclination of the arm enables it to clear an accessory device located on the rear surface 16.

The upper and lower articulation arms are connected together by an elbow joint. The elbow joint includes a bearing shaft 46 riding in a flanged bearing 48 and connected by a bolt 88 to the upper articulation arm 34. Rotation of the elbow joint is facilitated by a thrust washer 72 having a lubrication surface and located between the lower and upper articulation arms. In a constructed embodiment the elbow joint is allowed to turn freely but has two detent positions in which the joint can be locked in a fixed position. This is provided by a locking mechanism, including a lock release mechanism 44 with a lock button 42, a pair of spring-loaded lock pins 50, and a pair of spring retainers 52 which retain two springs (not shown) in the locking mechanism. Whenever the articulation arms are oriented parallel to each over in an overlapping position, or parallel to each other and in an extended position, the two lock pins 50 snap into holes in the upper articulation arm, locking the two arms in position. To release this locking mechanism the lock button 42 is pushed to rotate the lock release mechanism 44 against the force of the two springs. As the lock release mechanism rotates a cammed surface pulls the lock pins downward and out of engagement with the upper articulation arm. The elbow joint can again turn freely until one of the detent positions are engaged again. This mechanism allows the articulating mechanism to be operated as either a three-pivot mechanism or a two-pivot mechanism, at the option of the user. When the elbow is locked to operate the articulation mechanism as a two-pivot mechanism, it can form either a mechanism with both pivot axes aligned (if the arms are of the same length) and centrally located over the cart, or with the pivot axes as widely spaced as possible and the display in a fully extended position. When locked with the two articulation arms aligned and overlapping, the monitor can be located in its nominal “home” position with the display screen facing forward and the elbow extending directly to the rear of the cart. The weight of the monitor is then centered above the center of the cart, where it should be when the cart is being moved. It will be appreciated that a locking mechanism could be located at two or three of the pivot joints of the articulating mechanism to lock several or all of the joints when the cart is being moved or transported.

At the other end of the upper articulation arm 34 the monitor tilt and swivel base 38 is pivotally mounted to the upper arm by a similar flanged bearing 82, bearing shaft 84 and bolt 86. A thrust washer 76 is located between the joint surfaces of the base 38 and the arm 34. This joint is not allowed to rotate continuously as is the elbow joint, but is only permitted to turn in one circle of approximately 360°. In a constructed embodiment a pin extends upward from the upper arm 34 and engages a circular trough formed in the die-cast monitor base 38. This pin and trough allow the monitor to turn 80° in one direction from its nominal forward position and 270° in the other direction, for a total pivotal rotation of approximately 350°. This restriction on continuous rotation prevents the display cables and cords inside the arms from becoming overly twisted due to continuous pivoting of the monitor 20.

The monitor base 38 is seen to be formed to enable the monitor to rock upward and downward in the same manner as a conventional computer monitor base mount.

In a constructed embodiment the monitor can be moved laterally to the left and right of its nominal center position by 11 inches in each direction. The articulation mechanism in that embodiment also enables the monitor to be moved forward 7 inches and rearward 11 inches.

To enable the user to easily move the display device to a different position with one hand, a handle 100 is provided on the front of the display. The handle can be either formed as a part of or attached to the display bezel 110 as shown in FIG. 5. The handle 100 is shown in a plan view in FIG. 6. When the handle 100 is formed as a separate part from the display case or bezel 110, it is preferably formed as a hollow molded part of the same material and color as the display case or bezel. In a constructed embodiment the handle is formed from an ABS plastic material. In a preferred embodiment the handle is attached to the front of display both mechanically and adhesively. In a constructed embodiment the handle is bolted to the bezel from the inside of the bezel and is also bonded to the bezel with a urethane adhesive.

Alternative ways of controlling the display position are shown in FIGS. 7a-7e. In FIG. 7a the front handle 100 is replaced by two handles 106 and 108 on the sides of the display. These handles enable the operator to exert firmer control over the monitor when repositioning it. However, the operator must generally use both hands when trying to move the monitor forward without pivoting it, which is not the case with the front handle 100 of the preferred embodiment. Instead of handles it is also possible to use knobs 112, 114 as illustrated by FIG. 7b. Another alternative is to use flaps 116, 118 on the sides of the display as shown in FIG. 7c. These flaps may be formed to give a more artistic appearance to the monitor, and may be formed with concave rear surfaces that can be engaged when the flaps are gripped. Another alternative is to use two handles 122, 124 on the front of the display as shown in FIG. 7d. This embodiment presents the same difficulty as the embodiments of FIGS. 7a-7c, in which the user is unable to apply a forward or rearward force to the center of the monitor. In FIG. 7e two knobs 128 and 128 are used on the front of the display.

Other variations will also occur to those skilled in the art. For example, the ends of the handle 199 can be extended beyond the attachment point to the bezel and beyond the sides of the display. The extended ends can be curved to wrap around the sides of the monitor, thereby providing both a grip in the front center of the monitor and grips on the sides.

In use the operator can grab the handle with one hand and move or pivot the monitor easily with one hand to a different viewing position. The front handle 100 has been found to be the preferred position for the handle for swiveling the monitor, moving it from side to side or front to rear, and rocking the monitor to face upward or downward.

What is claimed is:

1. An ultrasonic diagnostic imaging system comprising:
a cart in which electronic components of the system are located;
a control panel connected to the cart;
a display articulating mechanism mounted on the cart;
an image display mounted on the articulating mechanism; and
a handle located on the image display,
wherein the position of the image display relative to the cart is changed by changing the position of the articulating mechanism by manually applying a force to the image display handle.

2. The ultrasonic diagnostic imaging system of claim 1, wherein the handle is located on the front of the image display.

3. The ultrasonic diagnostic imaging system of claim 2, wherein the image display comprises a CRT display.

4. The ultrasonic diagnostic imaging system of claim 2, wherein the image display comprises a flat panel display.

5. The ultrasonic diagnostic imaging system of claim 1, wherein the display articulating mechanism further comprises a first articulating joint having a vertical pivot axis and a second articulating joint having a horizontal pivot axis,
wherein the application of a manually applied force to the handle enables the display to be articulated about the first or second pivot axes.

6. The ultrasonic diagnostic imaging system of claim 1, wherein the articulating mechanism and display exhibit a nominal home position,
wherein the application of a manually applied force to the handle enables the display to be articulated to the left, to the right, to the front, and to the rear of the nominal home position.

7. The ultrasonic diagnostic imaging system of claim 1, wherein the image display comprises a display device housed in an outer enclosure,
wherein the handle is affixed to the outer enclosure by both a mechanical fastener and an adhesive.

8. The ultrasonic diagnostic imaging system of claim 7, wherein the outer enclosure further includes a front bezel,
wherein the handle is affixed to the front bezel.

9. The ultrasonic diagnostic imaging system of claim 1, wherein the handle further comprises a first handle affixed to one side of the image display, and a second handle affixed to the opposite side of the image display.

10. The ultrasonic diagnostic imaging system of claim 9, wherein the first and second handles comprise knobs.

11. The ultrasonic diagnostic imaging system of claim 9, wherein the first and second handles comprise graspable flaps.

12. The ultrasonic diagnostic imaging system of claim 2, wherein the handle further comprises a first handle located to one side of the image display, and a second handle located to another side of the image display.

13. The ultrasonic diagnostic imaging system of claim 12, wherein the handles comprise knobs.

14. The ultrasonic diagnostic imaging system of claim 2, wherein the handle extends beyond the lateral sides of the image display.

15. The ultrasonic diagnostic imaging system of claim 14, wherein the handle extends around the sides of the image display.
